(19) United States
(12) Patent Application Publication
(10) Pub. No.: US 2007/0128675 A1
(43) Pub. Date: Jun. 7, 2007

(54) METHOD OF CANCER SCREENING AND METHOD OF CANCER TREATMENT

(76) Inventor: John R. Woodward, Dallas, TX (US)

Correspondence Address: Michael A. O'Neil;Michael A. O'Neil, P.C., Suite 820, 5949 Sherry Lane, Dallas, TX, 75225, US

(21) Appl. No.: 11/622,090

(22) Filed: Jan. 11, 2007

Related U.S. Application Data
(62) Division of application No. 10/946,213, filed on Sep. 21, 2004.

(51) Int. Cl. G01N 33/574; A61B 5/05

(52) U.S. Cl. 435/7.23; 600/411

(57) ABSTRACT

A method of cancer screening comprising the steps of administering the Blood CA 27,29 testing procedure; if the result is positive administering a mammogram; if the result is positive administering an needle biopsy; if the result is positive administering a PET scan; if the result is positive administering a blood tumor cell count. If all of the foregoing steps are positive, the cancer is treated by applying imiquimod transdermally to rotating sites, preferably by mixing ALDARA™ 5% cream with an equal amount of H base cream™.

### CROSS-REFERENCE TO RELATED APPLICATION

This application is a divisional application of application Ser. No. 10/946,213 filed Sep. 21, 2004, currently pending, the entire contents of which are incorporated herein by reference.

### TECHNICAL FIELD

This invention relates generally to the detection and treatment of cancers, and more particularly to a method of screening for and a method of treating duct cell cancer of the breast, squamous cell cancer of the uterine cervix, and anal cancer.

### BACKGROUND AND SUMMARY OF THE INVENTION

As is well known, various technologies are available to the medical profession for use in determining the presence of cancers in patients. Included are x-ray studies, magnetic resonance imaging (MRI) studies, CT scans, as well as studies of various body fluids such as blood, urine, etc. Potential sites for colon cancer, for example, can be investigated utilizing electro-optical technologies. In some cases needle biopsy or exploratory surgery is necessary to confirm either the presence or absence of suspected cancer.

Various techniques for treating cancers are also well known. Certain cancers can be surgically removed, whereas other cancers require radiation therapy, chemotherapy, or combinations of radiation therapy and chemotherapy. Other cancers are susceptible to control using one or more drug therapies.

The present invention comprises a method of cancer screening and a method of cancer treatment which has proven successful in controlling epidermal cancers including, but not limited to, duct cell breast cancer, cervical squamous cancer, and anal cancer. In accordance with the broader aspects of the invention, a method of cancer screening involves a series of testing procedures each more expensive than the one before. Only when results of each of the testing procedures are positive is the presence of cancer confirmed. The invention further comprises a method of cancer treatment wherein the drug imiquimod is administered transdermally.

### BRIEF DESCRIPTION OF THE DRAWING

A more complete understanding of the present invention may be had by reference to the following Detailed Description when taken in connection with the accompanying Drawings, wherein:

FIG. 1 is a flowchart illustrating initial steps in the cancer screening method of the present invention; and

FIG. 2 is a flowchart illustrating subsequent steps in the cancer screening method of the present invention.

### DETAILED DESCRIPTION

### Introduction

The following example describes a method of detecting and treating duct cell breast cancer. However, the present invention is equally applicable to other epidermal cancers, such as squamous cancer of the uterine cervix and anal cancer.

### Example

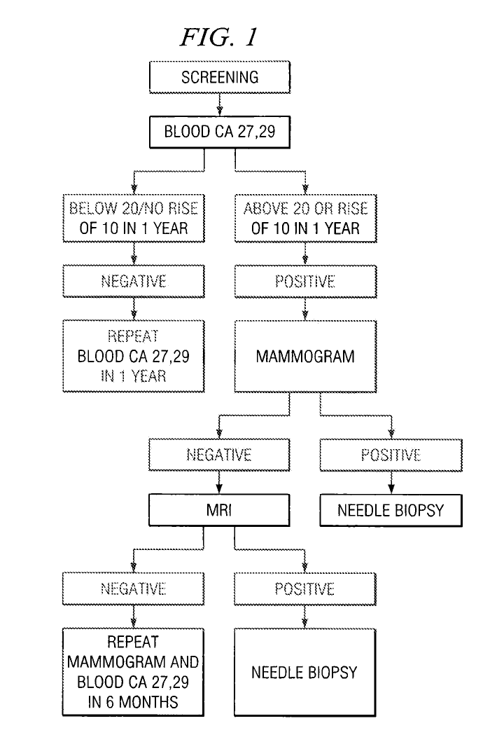

Referring to the Drawings, and particularly to FIG. 1 thereof, the early steps in the method of cancer screening of the present invention are shown therein. Screening begins with administration of the testing procedure known as Blood CA 27,29. The Blood CA 27,29 testing procedure has heretofore been utilized in monitoring the results of existing cancer treatment procedures. However, the Blood CA 27,29 procedure has not heretofore been used for cancer screening.

If the number comprising the results of the Blood CA 27,29 procedure is less than 20, and if there has been no increase in the number comprising the result of the Blood CA 27,29 testing procedure of ten (10) or more in the immediately preceding year, the result of the Blood CA 27,29 testing procedure is considered to be negative. The patient is then scheduled for follow-up testing utilizing the Blood CA 27,29 procedure in one year.

If the number comprising the result of the Blood CA 27,29 procedure is 20 or above, or if there has been an increase of 10 or more in the number comprising the result of the CA 27,29 testing procedure in the immediately preceding year, the result of the Blood CA 27,29 procedure is considered to be positive. In that event a mammogram testing procedure is administered. If the result of the mammogram testing procedure is negative, an MRI testing procedure is administered. If the result of the MRI testing procedure is negative, both the mammogram testing procedure and the Blood CA 27,29 testing procedure are re-administered in six months time. Conversely, if either the mammogram testing procedure is positive or the MRI testing procedure is positive, a needle biopsy of the identified lesion is performed.

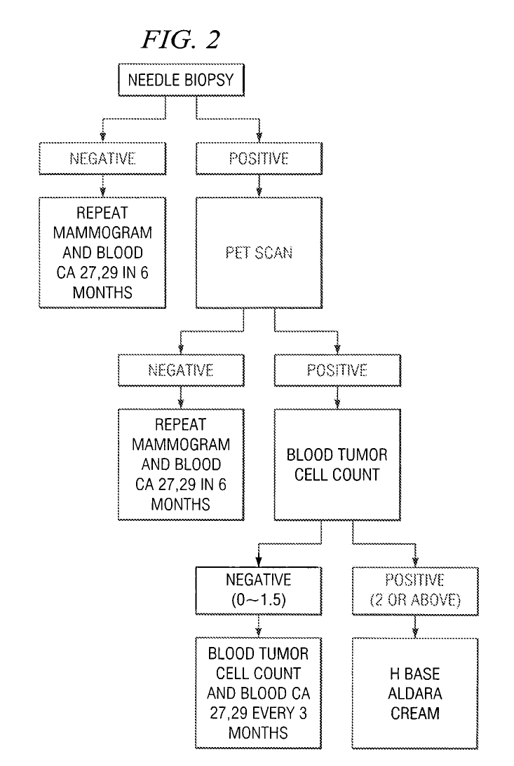

Referring to FIG. 2, if the results of the needle biopsy testing procedure are negative, both the mammogram testing procedure and the Blood CA 27,29 testing procedure are re-administered in six months. If the needle biopsy testing procedure is positive, a positron emission tomography (PET) scan testing procedure is administered. If the result of the PET scan testing procedure is negative, the mammogram testing procedure and the Blood CA 27,29 testing procedure are re-administered in six months. If the result of the PET scan testing procedure is positive, a blood tumor cell count testing procedure is administered. If the result of the blood tumor cell count testing procedure is negative, that is, if the number comprising the result of the blood tumor cell count testing procedure is between 0 and 1.5, the blood tumor cell count testing procedure and the Blood CA 27,29 testing procedure are administered at three month intervals. Conversely, if the blood tumor cell count testing procedure is positive, that is, if the number comprising the result of the blood tumor cell count testing procedure is two or above, the cancer treatment procedure of the present invention is administered.

The cancer treatment procedure of the present invention comprises the transdermal administration of the drug imiquimod. Imiquimod is commercially available from 3M Pharmaceuticals under the trademark ALDARA™. In accordance with the present invention, ALDARA™ cream 5% is mixed at a 1:1 ratio with H base cream. The ingredients of H base cream are:
water, glycerin, canola oil, stearic acid, cetyl alcohol, PEG-100 stearate, glyceryl stearate, dimethicone, magnesium aluminum silicate, propylene glycol, triethanolamine, polysorbate 60, xanthan gum, bitter almond kernel oil, aloe vera, grape seed extract, wheat germ oil, vitamin E acetate, vitamin A palmitate, Vitamin C palmitate, tetrasodium EDTA, potassium sorbate, diazolidinyl urea. H base cream is a proprietary product produced by Professional Compounds Centers of America and licensed by it.

The mixture of imiquimod and H base cream as described above is administered transdermally, preferably by mixing ¼ cc ALDARA™ 5% cream with ¼ cc H base cream and applying the resulting mixture to various locations, the inner thigh, abdomen, hip arms, etc., of the patient. Various sites of administration prevent any possible skin irritation. The foregoing amount of the mixture of ALDARA™ 5% cream and H base cream is applied daily, and the results of the procedure are periodically monitored utilizing the Blood CA 27,29 testing procedure.

Although preferred embodiments of the invention have been illustrated in the accompanying Drawings and described in the foregoing Detailed Description, it will be understood that the invention is not limited to the embodiments disclosed, but is capable of numerous rearrangements, modifications, and substitutions of parts and elements without departing from the spirit of the invention.

1. A method of cancer screening comprising the step of administering the Blood CA 27,29 testing procedure;
the subsequent step of administering an MRI;
the subsequent step of administering a needle biopsy; and
the subsequent step of administering a PET scan.

2. The method of cancer screening according to claim 1 including the subsequent step of administering a blood tumor cell count.
